(19) United States
(12) Patent Application Publication
(10) Pub. No.: US 2017/0098061 A1
(43) Pub. Date: Apr. 6, 2017

(54) Method of Receiving and Handling a Plurality of Clinical Samples for Reporting a Sum of Diagnostic Results for Each Sample

(71) Applicant: Medical Diagnostic Laboratories LLC, Hamilton, NJ (US)

(72) Inventors: Martin Adelson, Robbinsville, NJ (US); Eli Mordechai, East Winsdor, NJ (US)

(73) Assignee: Medical Diagnostic Laboratories, LLC, Hamilton, NJ (US)

(21) Appl. No.: 15/380,264

(22) Filed: Dec. 15, 2016

Related U.S. Application Data
(63) Continuation-in-part of application No. 11/343,826, filed on Jan. 31, 2006.
(60) Provisional application No. 60/654,729, filed on Feb. 18, 2005.
(60) Provisional application No. 60/654,485, filed on Feb. 18, 2005.
(60) Provisional application No. 60/651,688, filed on Feb. 10, 2005.

(51) Int. Cl. G06F 19/00; C12Q 1/70; C12Q 1/68

(52) U.S. Cl. CPC G06F 19/366; C12Q 1/689; C12Q 1/6895; C12Q 1/705

(57) ABSTRACT

A method is provided for receiving and handling a plurality of clinical samples and managing information associated therewith for generating and reporting any of a plurality of different diagnostic results from each sample in a timely manner, particularly within about thirty (30) hours. Methods described comprise, for example, receiving a plurality of single gynecological swab samples, each having identity and test requisition information associated therewith, wherein the test requisition information indicates a test for at least one causative agent, from a choice of a plurality of agents (for example, between about 5 and about 25 different microbiological agents) and managing information associated therewith for generating and reporting any of a plurality of different diagnostic results for each sample.

### CROSS REFERENCE TO RELATED APPLICATIONS

The present application is a continuation-in-part of application Ser. No. 11/343.826, entitled “A Method of Receiving and Handling a Plurality of Clinical Samples for Reporting a Sum of Diagnostic Results for Each Sample,” filed on Jan. 31, 2006, the entire contents of which are hereby incorporated by reference, and claims the benefit of said application under 35 U.S.C. 120. Additionally, the present application claims benefit, under 35 U.S.C. 119(e), to U.S. Provisional Application No. 60/651,688, entitled “A Method and Kit for the Collection and Maintenance of the Detectability of a Plurality of Microbiological Species in a Single Gynecological Sample,” filed on Feb. 10, 2005, the entire contents of which are hereby incorporated by reference. Additionally, the present application claims benefit, under 35 U.S.C. 119(e), to U.S. Provisional Application No. 60/654,485, entitled “Integrated Method for Collection and Maintenance of Detectability of a Plurality of Microbiological Agents in a Single Clinical Sample and for Handling a Plurality of Samples for Reporting a Sum of Diagnostic Results for Each Sample,” filed on Feb. 18, 2005, the entire contents of which are hereby incorporated by reference. Also, the present application claims benefit, under 35 U.S.C. 119(e), to U.S. Provisional Application No. 60/654,729, entitled “A Method of Receiving and Handling a Plurality of Clinical Samples for Reporting a Sum of Diagnostic Results for Each Sample,” filed on Feb. 18, 2005, the entire contents of which are hereby incorporated by reference.

### BACKGROUND OF THE INVENTION

1. Field of the Invention

The present invention is in the field of clinical diagnostic services. The invention is particularly related to managing information concerning a plurality of clinical samples, each having identity and test requisition information associated therewith, for generating and reporting a sum of diagnostic results for each sample.

2. Description of the Related Art

Rapid and accurate identification of causative agents of a myriad of different human pathophysiological conditions is a paramount requisite to effective treatment. Clinical diagnostics provide an essential aid to the physician for the diagnosis and monitoring of numerous gynecological pathologies and infectious diseases. A physician may suspect a particular causative agent upon physical examination. However, certain symptoms may be characteristic of a plethora of different causative agents. Diagnostic kits are available, for example, capable of detecting specific species, at most several. However, current products and services are inadequate to identify causative agents of gynecological disorders, for example, in a timely manner or are inoperable under clinical circumstances.

Methods are accordingly needed for receiving and handling a plurality of single gynecological swab clinical samples, each having identity and test requisition information associated therewith, wherein the test requisition information indicates a test for at least one causative agent from a plurality of listed agents, and managing information associated therewith for reporting a sum of diagnostic results for each sample.

### BRIEF SUMMARY OF THE INVENTION

The present invention is directed to a method of receiving and handling a plurality of clinical samples and managing information associated therewith for generating and reporting any of a plurality of different diagnostic results from each sample in a timely manner, particularly within about thirty (30) hours. Methods described herein comprise, for example, receiving a plurality of single gynecological swab samples, each having identity and test requisition information associated therewith, wherein the test requisition information indicates a test for at least one causative agent, from a choice of a plurality of agents (for example, between about 5 and about 25 different microbiological agents) and managing information associated therewith for generating and reporting any of a plurality of different diagnostic results for each sample.

### BRIEF DESCRIPTION OF THE DRAWINGS

FIG. 1 is an example of a Test Requisition Form.

FIG. 2 displays validation data for Bacteroides fragilis wherein the PCR amplicon is 842 by in which each sample was independently inoculated and extracted in triplicate. Lanes 2-4 represent detectability after storage at room temperature for zero days. Lanes 5-7 represent detectability after storage at room temperature for one day. Lanes 8-10 represent detectability after storage at room temperature for two days. Lanes 11-13 represent detectability after storage at room temperature for three days. Lanes 14-16 represent detectability after storage at room temperature for four days. Lanes 17-19 represent detectability after storage at room temperature for five days. Lanes 20 and 21 are the positive and negative controls, respectively.

FIG. 3 displays validation data for Mobiluncus mulieris wherein the PCR amplicon is 1015 by in which each sample was independently inoculated and extracted in triplicate. Lanes 2-4 represent detectability after storage at room temperature for zero days. Lanes 5-7 represent detectability after storage at room temperature for one day. Lanes 8-10 represent detectability after storage at room temperature for two days. Lanes 11-13 represent detectability after storage at room temperature for three days. Lanes 14-16 represent detectability after storage at room temperature for four days. Lanes 17-19 represent detectability after storage at room temperature for five days. Lanes 20 and 21 are the positive and negative controls, respectively.

FIG. 4 displays validation data for Candida albicans.

FIG. 5 displays validation data for Candida glabrata.

FIG. 6 displays validation data for Candida parapsilosis.

FIG. 7 displays validation data for Candida tropicalis.

FIG. 8 displays validation data for Chlamydia trachomatis.

FIG. 9 displays validation data for Gardnerella vaginalis.

FIG. 10 displays validation data for Haemophilis ducreyi.

FIG. 11 displays validation data for HSV-1.

FIG. 12 displays validation data for HSV-2.

FIG. 13 displays validation data for Trichomonas vaginalis.

FIG. 14 displays validation data for Ureaplasma urealyticum.

### DETAILED DESCRIPTION

The present invention Unless defined otherwise, all technical and scientific terms used herein have the same meaning as is commonly understood by one of skill in the art to which this invention belongs. All publications and patents referred to herein are incorporated by reference.

Physicians are generally faced with observing patients' symptoms, obtaining biological samples, and ordering clinical diagnostic tests to determine the identity of causative agents which mediate pathological conditions. Since methods of treatment of pathophysiological conditions are intimately related to the identity of the causative agent(s) of the condition, rapid and accurate identification and reporting of the causative agent(s) is of paramount importance to the practice of medicine today. The present invention enables the accurate and rapid reporting of the detection of any of a plurality of biological agents from each clinical sample.

The present invention is fundamentally a method for receiving and handling a plurality of clinical samples and managing information associated therewith. The flow of information and reporting of results from a clinical laboratory is a fundamental aspect of the present invention. The present invention enables the accurate and rapid reporting of the detection of any of a plurality of biological agents from each clinical sample. The present invention is also a method for receiving and handling a plurality of clinical samples and managing information associated therewith to effect rapid diagnostic testing for any of a plurality of agents in each sample. Although the invention defined by the claims appended hereto are not necessarily so limited, preferred methods of the invention are for handling a plurality of single gynecological swabs (e.g., each from a different patient) and managing information associated therewith.

A basic embodiment of the method of the present invention involves a rapid method of handling a plurality of single gynecological swab samples and managing information associated therewith for reporting any of a plurality of different diagnostic results for each sample within about fifty (50) hours of receiving the sample (preferably within forty eight (48) hours, more preferably within about thirty (30) hours, most preferably within about twenty four (24) hours). Single gynecological swab samples each have identity and test requisition information associated therewith, wherein the test requisition information indicates a test for at least one causative agent, from a choice of a plurality of agents (for example, between about 5 and about 25 different microbiological agents). The term “causative agent” as used herein refers to biological entities that mediate disease conditions, including, but not limited to, microorganisms, e.g., bacteria fungi, and viruses. Preferred agents, referred to herein as causative agents, include but are not limited to microbiological species associated with pathological gynecological conditions, for example, collected in a single swab specimen (clinical sample). Causative agents referred to herein include, but are not limited to Bacteroides fragilis, Candida albicans, Candida glabrata, Candida parapsilosis, Candida tropicalis, Chlamydia trachomatis, Gardnerella vaginalis, Haemophilis ducreyi, Herpes simplex virus subtype 1 (HSV-1), Herpes simplex virus subtype 2 (HSV-2), Human papillomavirus (HPV), Mobiluncus mulieris, Mobiluncus curtisii, Molluscum contagiosum Virus, Mycoplasma genitalium, Mycoplasma hominis, Neisseria gonorrhoeae, Treponema pallidum, Trichomonas vaginalis, Ureaplasma urealyticum, and Streptococcus agalactiae (Group B Streptococcus).

In some embodiments the microbiological species comprise at least two, three, four, five, ten or fifteen of the following: Candida dubliniensis, Candida lusitaneae, Atopobium vaginae Lymphogranuloma venereum, erythromycin-resistant Streptococcus agalactiae clindamycin-resistant Streptococcus agalactiae, Bacteroides fragilis, Candida albicans, Candida glabrata, Candida parapsilosis, Candida tropicalis, Chlamydia trachomatis, Gardnerella vaginalis, Haemophilus ducreyi, Herpes simplex virus subtype 1, Herpes simplex virus subtype 2, Human papillomavirus (HPV), Mobiluncus mulieris, Mobiluncus curtisii, Molluscum contagiosum Virus, Mycoplasma genitalium, Mycoplasma hominis, Neisseria gonorrhoeae, Treponema pallidum, Trichomonas vaginalis, Ureaplasma urealyticum, Streptococcus agalactiae, Candida krusei, HPV-16, HPV-18, HPV-31, HPV-33, HPV-35, HPV-39, HPV-45, HPV-51, HPV-52, HPV-56, HPV-58, HPV-59, HPV-66, HPV-68, HPV-30 6/11, HPV-42, HPV-43, or HPV-44. In some embodiments the microorganisms are at least five selected from the group consisting of the immediately preceding list of species.

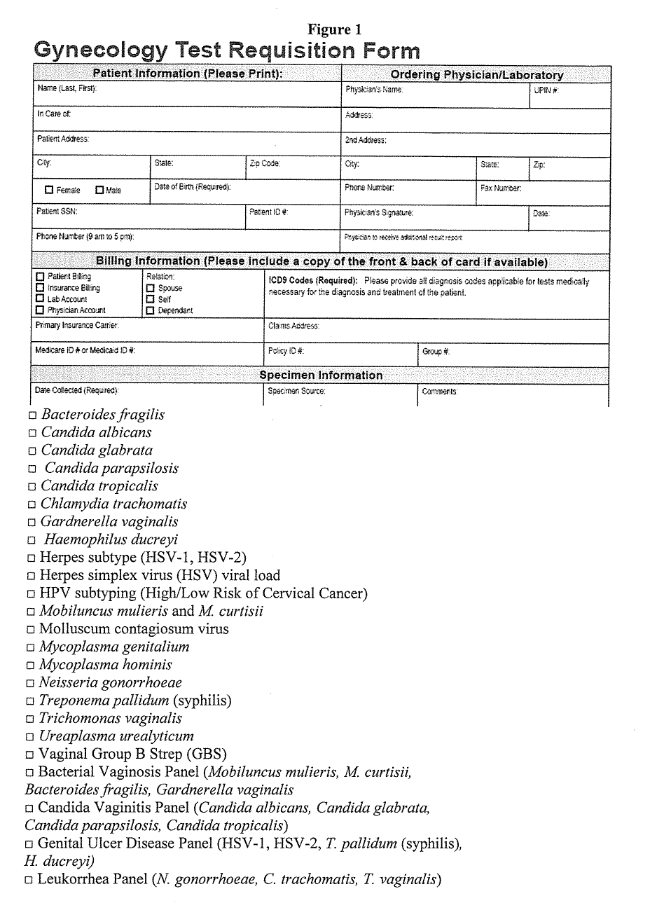

The term “clinical sample,” as used herein, refers to biological samples known in the art. “Clinical sample” includes, for example, but is not limited to a gynecological swab sample. Particularly, the method of the present invention provides materials for the collection and maintenance of a “snapshot” of a gynecological environment for the detectability of a plurality of species of microbiological agents in a single gynecological sample. Methods described herein also comprise (1) providing a transport media in a resealable container, a sterile swab, and instructions for preparation and handling of a gynecological sample and a written indication of the detectability of the plurality of species, e.g., a test requisition form (see FIG. 1), (2) receiving the completed gynecological sample in a package with a completed test requisition form, and (3) handling a plurality of clinical samples and managing information associated therewith for reporting any of a plurality of different diagnostic results for each sample in a timely manner. Reporting may be accomplished by means of facsimile to an attending physician who ordered the test(s), for example, or other means, electronic or otherwise, e.g., posting on a private-access internet web site, including all means that are usual and customary in the health-care industry.

Clinical samples are generally labeled or otherwise clearly associated, e.g., packaged, with information that distinctly identifies the origin, source and/or destination for the results for each sample. Each sample is associated with an identifier, e.g., a patient's name, date of birth, and/or social security number, for example, or information otherwise provided by the source to indicate the distinct origin of each sample.

The present invention is not drawn to methods for the detection, identification or diagnosis, per se, of any particular microbiological species, or series of species, or disease condition. The tests, per se, however, whatever tests are used, are not relevant to the subject matter of the present invention. In contrast, methods described herein are specifically directed toward handling a plurality of clinical samples and managing information associated therewith. Particularly, methods of the present invention are for managing samples, materials, and information related to the samples proximal in time, i.e., before, during, and after, a determination of any of a plurality of different possible diagnostic results for each sample and reporting the results. Methods described herein are preferably preceded by the collection and maintenance of detectability of a plurality of species of microbiological agents selected from the group consisting of bacteria, fungi, and viruses, in a single gynecological sample comprising providing transport media in a resealable container, a sterile swab, and instructions for preparation and handling of a gynecological sample and an indication of the detectability of the plurality of species.

Microbiological agents that are causative or are otherwise associated with gynecological disorders are preferred. Since many different species of microbiological agents mediate, or are associated with, or are indicative of gynecological disorders, the present invention provides a means for handling a plurality of clinical gynecological swab samples and managing information associated therewith in the process leading up to the identification of at least one causative agent in each sample and reporting the results representative of the ambient population of microbiological agents in each sample at the time each sample was taken. Particularly, the method of the present invention enables a “snapshot” of details corresponding to a single gynecological sample, within a plurality of samples, to be provided in a valuable period of time by means of information management. A single gynecological swab sample is generally received in a liquid universal transport media in which viability of a plurality of organisms, e.g., bacteria, fungi, and viruses, can be sustained under normal conditions without refrigeration for at least 48 hours. A single gynecological swab sample is generally received in transport media, between about 1 ml and about 5 ml, for example, in a resealable container along with a test requisition form.

Receiving a Plurality of Single Gynecological Swab Samples

A “plurality of samples” is an inclusive term which refers to a plurality of single samples from different patients. A “plurality” of samples generally refers to a substantial number of biological samples received by a clinical lab within a twenty four (24) hour period, for example. A plurality of samples, however, as used herein may refer to as few as several samples, e.g., about ten (10), or about five thousand (5,000) samples, for example, to be processed. Each sample has identity and test requisition information associated therewith, wherein the test requisition information indicates a test for at least one causative agent, from a choice of a plurality of agents, from a list of between about 5 and about 25 different microbiological agents, for example. For the purpose of illustration of the complexity of information associated with a plurality of single gynecological swabs and test requisition information associated therewith, each sample with a test requisition form which indicates the detectability of twenty (20) different agents, for example, has the possibility of about 400 different diagnostic results, for that single sample. This, combined with the fact that a plurality of samples (e.g., 500) are received to be processed together, that day, illustrates the complexity of information associated therewith to be managed in order to handle the plurality of clinical samples for reporting any of a plurality of different diagnostic results for each sample in a timely manner. The current invention is particularly directed to methods wherein the test requisition information indicates a test for at least one (1), preferably at least two or three (2 or 3), e.g., between 4 and about 6, causative agents from a choice (list) of a plurality of agents. Embodiments of the present invention include embodiments, for example, wherein the plurality of species comprise at least one species selected from the group consisting of Molluscum contagiosum Virus, Mycoplasma genitalium, and Mycoplasma hominis.

The information in the system, i.e., the identity of the sample (e.g., sample identifier or identification tag) and test requisition information, i.e., tests specifically requested to be performed on that sample, is processed to designate a test on each sample for at least one causative agent. Accordingly, methods described herein comprise entering identity and test requisition information associated with each sample into a system to create a requisition file for each sample. The term “system” as used herein refers generally to a system of recording and managing information, a computer implemented information management system to manage the flow of information and, in certain embodiments, to control instrumentation, throughout the process of the present invention. This system is preferred, but, however, is not required. A computer is generally employed to receive the identity and test requisition information associated with each sample. The information may be entered manually into a server, for example, to create a test requisition file for each sample which comprises the sample information and the test requisition information. A listing, file for example, of the identity of all samples for each test is created. If twenty different tests are to be performed (for twenty different pathological agents), for example, twenty different lists of sample identifiers are created. If a certain sample will be subject to three different tests, for example, that sample identifier will be on at least three separate lists corresponding to those three different tests. In some embodiments of the present invention a computer implemented system performs calculations and/or controls instrumentation.

Nucleic Acid is Extracted from Each Sample

Established procedures for DNA extraction are used (see Example II). In brief, swabs are thoroughly mixed in the transport media contained within the transport vials. 470 μl of transport media is mixed with 25 μl of 10% sodium dodecyl sulfate (SDS), and 12 μl of freshly prepared DNase-free proteinase-K (10 mg/ml), then incubated for 2 hours at 55° C. DNA is phenol:chloroform:isoamyl alcohol extracted and recovered by ethanol precipitation. DNA is pelleted, dried in a speed vacuum, and resuspended in 20 μl TE buffer. DNA concentration is calculated by absorbance 260/280 readings and is adjusted to 0.2 μg/μl prior to PCR analysis. Quantitation, however, is preferred using a fluorometer such as one available from Turner BioSystems, Inc., Sunnyvale, Calif.

The nucleic acid from each sample is diluted to about 200 ng/μl, for example, with water, for example, to provide a standardized primary nucleic acid solution corresponding to each sample. An aliquot of nucleic acid from each sample is dispensed into a separate individual vessel to create a secondary sample corresponding to each designated test on each sample. A general supply of master reagent mix, e.g., real-time PCR mix, for each test for each different causative agent is prepared. An aliquot of each master reagent mix is combined with each corresponding secondary nucleic acid sample for each test to produce a diagnostic test reaction for each secondary sample. Each reaction is incubated and preferably monitored in real-time. The presence or absence of a certain product of each reaction to produce a result is determined. The result of each reaction is recorded in the system. The result of each reaction derived from each primary solution is combined into the requisition file for each sample on the system, thereby identifying at least one causative agent in each sample, and the results of the identification are reported.

A rapid method of handling a plurality of clinical samples and managing information associated therewith for identifying at least one causative agent in each sample and reporting results comprises receiving a plurality of samples, each having identity and test requisition information associated therewith wherein the test requisition information indicates a test for at least one causative agent, entering the information into a system to create a requisition file for each sample, processing the information to designate a test on each sample for at least one causative agent, dispensing an aliquot corresponding to each sample into an individual vessel to create a secondary sample for each designated test, assembling a general supply of master reagent mix for each test for a different causative agent, combining an aliquot of each master reagent mix with each corresponding secondary sample for each test to produce a diagnostic test reaction for each secondary sample, incubating each reaction, determining the presence or absence of a certain product of each reaction to produce a result, recording the result of each reaction, combining the result of each reaction derived from each primary sample into the requisition file for each sample on the system, thereby identifying at least one causative agent in each sample, and reporting the results of the identification.

A preferred method of handling a plurality of clinical samples and managing information associated therewith for identifying at least one causative agent in each sample and reporting results comprises receiving a plurality of samples, each having identity and test requisition information associated therewith wherein the test requisition information indicates a test for at least one causative agent, entering the information into a system to create a requisition file for each sample, extracting nucleic acid from each sample, quantitating the nucleic acid, diluting the nucleic acid from each sample to provide a standardized primary nucleic acid solution corresponding to each sample, processing the information in the system to designate a real-time PCR test on each nucleic acid for at least one causative agent, dispensing an aliquot of the primary solution from each sample into a separate individual vessel to create a standardized secondary nucleic acid sample for each designated test on each sample, assembling a general supply of master reagent mix for each test for a different causative agent, combining an aliquot of each master reagent mix with each corresponding secondary nucleic acid sample for each test to produce a diagnostic test reaction for each secondary sample, incubating each reaction, determining the presence or absence of a certain product of each reaction to produce a result, electronically recording the result of each reaction, in the system, combining the result of each reaction derived from each primary solution into the requisition file for each sample on the system, thereby identifying at least one causative agent in each sample, and reporting the results of the identification. Preferred methods of the invention described herein employ quantitating nucleic acid from each sample by means of a fluorometer. Methods of the present invention preferably comprise generation of diagnostic results by means of real-time PCR.

To Maxmize the Success of Clinical Diagnostic Methods Described Herein

A clinical diagnostic laboratory should be physically set up so that specimen separation and extractions occur in a separate room, using a “Class II Biohazard Safety Hood.” PCR preparation should occur in a separate room, within one of many PCR cabinets which are dedicated solely to PCR preparation. The PCR amplification should occur in thermocyclers located in an enclosed room. For post-amplification process of conventional PCR reactions, gel electrophoresis should be performed in yet another physically separate room. UV lights should be used in the PCR hoods and commercial solvents, such as DNAway (Molecular Bio Products, San Diego, Calif.), to decontaminate all work surfaces prior to and at the completion of any procedures occurring in that area. Sterile, disposable plasticware should be used wherever possible. All glassware should be autoclaved. All PCR reactions should be performed in individual closed tube systems as opposed to 96-well microtiter plates to eliminate cross contamination. Real-time PCR assays, for example, do not require gel electrophoresis and therefore eliminate post-amplification specimen handling. All technicians should only manipulate one specimen at a time. This means when a reagent is added to a batch of specimens, it occurs one tube at a time. The next patient's reaction tube is not opened until the previous patient's tube has been closed. Pipette tips used when dispensing reagents should be filtered to prevent aerosol contamination and are also replaced between all specimens. Reagents used during PCR preparation may be aliquoted into 1.5 ml microcentrifuge tubes, for example, as opposed to dispensation into stock bottles of greater volumes. This enables the laboratory to monitor potential contamination closely and discard any reagents, if ever necessary. The use of separate rooms is recommended to decontaminate an entire room if contamination is suspected. Positive and negative controls should be employed to assess false positives as well as false negatives. Uracil-N-glycosylase is recommended in every reaction to minimize, if not eliminate, any possible carry-over contamination.

Primers

Any pair of PCR primers may be employed in methods of the present invention that function to amplify target nucleic acids. The art of selection and synthesis of PCR primers in order to amplify a particular target sequence is indeed well-known to those of ordinary skill in the art. Typically, oligonucleotide primers are about 8 to about 50 nucleotides in length. Primers 12 to 24 nucleotides in length are preferred. Primer pairs that amplify particular nucleic acid molecules can be designed using, for example, a computer program such as OLIGO (Molecular Biology Insights, Inc., Cascade, Colo.). A biotin moiety, for example, is preferably attached to the 5′ end of one of the primers to facilitate sample preparation for “pyrosequencing,” a term which denotes the nucleotide sequencing method described in U.S. Pat. Nos. 6,210,891 and 6,258,568; Ronaghi et al., 1998, A sequencing method based on real-time pyrophosphate. Science 281:363-365; and Ronaghi, 2001, Pyrosequencing sheds light on DNA sequencing. Genome Research 11:3-11. Other entities, however, well known to those of skill in the art, may similarly be incorporated, integrated, or attached to one of the primers to facilitate the isolation of the resulting amplicon for pyrosequencing.

Real-Time PCR

Quantitative real-time PCR is a preferred method of amplification of a target nucleic acid. Products used to accomplish the methods are commercially available from several manufacturers including, but not limited, to Corbett Research (Mortlake, Australia), Cepheid (Sunnyvale, Calif.), BioRad (Hercules, Calif.), and Applied Biosystems (Foster City, Calif). The Corbett Research (Melbourne, Australia) Rotor-Gene™ 3000, for example, is a centrifugal, real-time DNA amplification system.

Validation studies exemplified herein are merely a general demonstration of the utility and value of the present invention, namely a method for the collection and maintenance of detectability of a plurality of species of microbiological agents in a single gynecological sample, in the grand scheme of clinical diagnostics. The legitimacy of the PCR method is not a relevant factor, as its utility as an invaluable molecular biological tool has already been well established in the international scientific literature through the publication of thousands of peer-reviewed articles. Particularly, molecular amplification of nucleic acids by means of PCR is well-known to those of ordinary skill in the art, i.e., the ability of the PCR method to detect genetic sequences specific to a target pathogen within a given clinical specimen. The Applicants particularly highlight, however, that the methods described herein, which comprise providing transport media in a resealable container to a physician, clinical lab, or medical institution, with instructions for preparing and handling a gynecological sample, along with a test requisition form which indicates the detectability of a plurality of species described herein, affect the ability of a physician, for example, to collect a single swab sample of a gynecological environment for the maintenance of detectability of a plurality of species of microbiological agents. Example assays designed to test sensitivity, specificity, interference and optimization were performed to validate the operability of the methods and materials described herein, as claimed. In other words, PCR methods or reagents employed to detect microbiological agents are not relevant to the scope of the subject matter of the claims appended hereto. In contrast, the present invention is solely drawn toward methods and certain materials for collection and maintenance of detectability of a plurality of species of microbiological agents in a single gynecological swab sample.

Sensitivity refers to a method's ability to detect very minute amounts of a substance or organism. The frequency of a positive test result in patients who have the disease the test is designed to detect, is expressed mathematically as follows:

$$Sensitivity = \frac{True Positives True \times 100}{Positives + False + False Negatives}$$

Sensitivity studies were initially performed by purchasing well-characterized, validated organisms from the American Tissue Culture Collection (ATCC, Manassas, Va.). The DNA of the virus, bacteria, or fungi is then extracted and quantitated. Standards of known concentrations are used to determine the assay's ability to detect varying concentrations of genetic material. The extracted DNA is serially diluted to concentrations of 1:10, 1:100, 1:1,000 and 1:10,000. By evaluating the presence of bands in these dilutions of known concentrations, the sensitivity of a particular test can be established. For real-time PCR assays, the fluorescence acquisition profile generated from the amplification of the serial dilutions is analyzed. A region encompassing the genetic target of the assay is generally subcloned into a vector system. Through quantitation of the vector and the optimization of the assay as described infra, as few as 10 genomic equivalents of the pathogen can be reproducibly detected.

Specificity studies were used to assess the quality of the primer selection for the assay by determining if their organisms' DNA will cross-react in any way leading to false positives. Initially as a theoretical test, primers are cross-referenced against the billions of other genetic sequences which have been deposited in the public databases by international researchers and any potential conflicts are avoided. Next as an experimental confirmation, the primers and probes are assayed for their inability to amplify dozens of other known bacterial, viral, and fungal organisms which have been identified as human pathogens. An aliquot of the characterized positive control is also spiked in a suspension consisting of the DNA of numerous other organisms to ensure that the particular pathogen target genome is in no way masked or inhibited by other genomic sequences.

Interference studies are used to determine if other substances inherent to the specimen type will interfere with detection by PCR. Certain effects, such as masking the organism's target to produce a false negative, or cross-reactivity to produce a false positive are analyzed. Characteristics, such as the microcosm of normal flora of the genital tract, the abundance of various proteins found in blood, and natural inhibitors commonly found in other body fluids, such as urine, can all have detrimental effects on the PCR process, unless accounted for during the initial processing and extraction procedures.

Optimization studies are the final step of the validation process. In these assays, the concentrations of various reagents are varied such as template DNA, MgCl₂, and primers, and probes as well as the temperature and duration of each step of the thermocycling parameters to improve the clarity of bands or the intensity of signals, as well as eliminate streaks, multiple banding, or haziness, which can impede the visualization of the PCR products or interpretation of real-time PCR results. A method is preferred wherein a progress of at least one reaction is optically monitored by means of the system and/or wherein the presence or absence of a product of at least one reaction is optically determined and electronically recorded by the system.

In one embodiment the present invention is directed to a method of handling a plurality of clinical samples and managing information associated therewith by a clinical laboratory for reporting a sum of diagnostic results for each sample comprising:
(a) receiving a plurality of primary gynecological swab samples, each primary gynecological swab sample in said plurality of gynecological swab samples having identity and test requisition information associated therewith wherein the test requisition information indicates a test for at least one microbiological causative agent from a plurality of microbiological causative agents listed in said test requisition, wherein said plurality of causative agents listed in said test requisition comprises Ureaplasma parvum.
(b) entering said requisition information into a computer system to create a requisition file for each said primary gynecological swab sample;
(c) processing said requisition information in said computer system to create a list of tests for each said microbiological causative agent;
(d) dispensing an aliquot corresponding to each said primary gynecological swab sample into one or more individual vessels, to create one or more secondary samples, each secondary sample corresponding to each said indicated test;
(e) assembling a general supply of master reagent mix for each said indicated test;
(f) combining an aliquot of a master reagent mix for each said indicated test with each corresponding secondary sample to produce a diagnostic test reaction for each said secondary sample;
(g) incubating each said diagnostic test reaction;
(h) performing a polymerase chain reaction (PCR) to determine the presence or absence of a certain product of each said diagnostic test reaction to produce a result;
(i) recording said result of each said PCR by means of said computer system;
(j) entering said result of each said PCR reaction derived from each said primary gynecological swab sample into said requisition file for each said sample on said computer system, thereby producing a sum of results of all tests for each said primary gynecological swab sample; and
(k) reporting said results to a physician within 24-48 hours of receiving said primary gynecological swab sample.

### ADDITIONAL REFERENCES

1. Adelson et al., 2005, Simultaneous detection of herpes simplex virus types 1 and 2 by real-time PCR and pyrosequencing. Journal of Clinical Virology 33:25-34. (manuscript published online on Nov. 14, 2004).

2. Trama et al., 2005, Detection of Candida species in vaginal samples in a clinical laboratory setting. Infectious Diseases in Obstetrics and Gynecology 13(2):63-67.

3. Trama et al., 2005, Detection and identification of Candida species associated with Candida vaginitis by real-time PCR and pyrosequencing. Molecular and Cellular Probes 19(2): 145-152.

4. Trama et al. Analyzing Candida albicans gene mutations that contribute to azole resistance by pyrosequencing. American College of Obstetricians and Gynecologists 52^{nd} Annual Clinical Meeting, May 1-5, 2004, Philadelphia, Pa.

5. Trama et al. Novel technique for identification of vulvovaginal candidiasis by real-time PCR and pyrosequencing. American College of Obstetricians and Gynecologists 52nd Annual Clinical Meeting, May 1-5, 2004, Philadelphia, Pa.

6. Adelson et al., Diagnosis of Neisseria gonorrhea, Chlamydia trachomatis, and Trichomonas vaginalis by real-time PCR. American College of Obstetricians and Gynecologists 52^{w}′ Annual Clinical Meeting, May 1-5, 2004, Philadelphia, Pa.

7. Mordechai et al., Prevalency of Candida species associated with Candida vaginitis in the United States. American Society of Microbiology 104thGeneral Meeting, May 23-27, 2004, New Orleans, La., Poster C-108.

8. Adelson et al., Development of a real-time PCR assay for the simultaneous detection of herpes simplex virus types 1 and 2 with confirmation by pyrosequencing technology. American Society of Microbiology 104th General Meeting, May 23-27, 2004, New Orleans, La., Poster C-273.

9. Naurath et al., Detection and quantification of Gardnerella vaginalis by real-time PCR. American College of Obstetricians and Gynecologists 53rd Annual Clinical Meeting. May 7-11, 2005, San Francisco, Calif.

10. Trama et al, Detection of molluscum contagiosum virus by real-time PCR and pyrosequencing. American Society of Microbiology 105th General Meeting, Jun. 5-9, 2005, Atlanta, Ga.

11. Feola et al., Detection of Ureaplasma urealyticum, Mycoplasma hominis, and Mycoplasma genitalium by real-time PCR and pyrosequencing. American Society of Microbiology 105^{th} General Meeting, Jun. 5-9, 2005, Atlanta, Ga.

12. Gygax et al., Erythromycin and clindamycin resistance in Group B Streptococcal clinical isolates. Presented by Dr. Martin E. Adelson at the 45^{th}ICAAC (Interscience Conference on Antimicrobial Agents and Chemotherapy) Meeting in Washington D.C. on Dec. 16, 2005.

13. Adelson et al., Evaluation of UTM-RT for the molecular detection of a plurality of OB/GYN related pathogens. Presented by Dr. Martin E. Adelson at the 45^{th} ICAAC (Interscience Conference on Antimicrobial Agents and Chemotherapy) Meeting in Washington D.C. on Dec. 17, 2005.

### EXAMPLES

### Example I

### Validation Studies

To determine if Copan UTM-RT media (Copan Diagnostics Inc., Corona, Calif) is suitable for the molecular amplifications diagnostic testing, the following pathogens were purchased from ATCC and detection assays were performed:

TABLE 1

| | | ATCC Catalogue |
|---|---|---|
| | Pathogen | Number |
| 1 | Bacteroides fragilis | 23745 |
| 2 | Candida albicans | 18804 |
| 3 | Candida glabrata | 2001 |
| 4 | Candida parapsilosis | 10233 |
| 5 | Candida tropicalis | 13803 |
| 6 | Chlamydia trachomatis | VR-901B |
| 7 | Gardnerella vaginalis | 14018 |
| 8 | Haemophilis ducreyi | 27721 |
| 9 | Herpes Simples Virus-1 | VR-1544 |
| 10 | Herpes Simples Virus-2 | VR-734 |
| 11 | Mobiluncus mulieris | 35243 |
| 12 | Mycoplasma hominis | 14027 |
| 13 | Neisseria gonorrhoeae | 27628 |
| 14 | Trichomonas vaginalis | 30246 |
| 15 | Ureaplasma urealyticum | 27618 |

### Simulation of a Positive Clinical Specimen

Pathogens were purchased from ATCC in a lyophilized pellet form. Each pellet was dissolved in five ml of TE-buffer (10 mM Tris, pH 7.5, and 1 mM EDTA) in case of bacteria or yeast liquid media (10 g of yeast extract, 20 g of peptone dissolved in 1 L of distilled water, pH 7) in case of fungi. Virus cultures were purchased from ATCC as two ml liquid cultures. Dilutions were subsequently prepared as follows:

TABLE 2

| | Concentration | | |
|---|---|---|---|
| | (Designation) | | |
| | 1:1 | 1:10 | |
| | (A) | (B) | 1:100 © |
| Original suspension | 600 μl | 60 μl | 6 μl |
| TE Buffer (Bacteria, virus) or Yeast | 0μ | 540 μl | 594 μl |
| Liquid Media (Fungi) | | | |

DNA was extracted from 500 μl of A, B, and C dilutions using standard laboratory phenol/chloroform/ethanol precipitation protocols. For positive controls, pathogen-positive clinical specimens were identified from the initial laboratory diagnostic tests and 500 μl of the corresponding original cervical swab media specimen was extracted. Previously validated real-time PCRs for each set of pathogens was performed on DNA extracted from Dilutions A, B, and C as well as the clinical samples. Rotor-Gene software calculated CTvalues for the three ATCC dilutions and the clinical specimens (Rotor-Gene 3000 instrument). The CT values of the dilutions were compared with that obtained for the clinical specimens and a “simulated dilution” was extrapolated for the subsequent studies of the Copan UTM-RT transport medium. Based upon these studies, the following was selected:

TABLE 3

| | | ATCC | TE Buffer | Overall |
|---|---|---|---|---|
| | | Resuspension | (Bacteria, Virus | Dilution |
| | ATCC | used in this | or Yeast Liquid | of |
| | Resuspension | experiment | Media (Fungi) | Pellet |
| Bactetria | 5 ml TE buffer | 5μ | 245 μl | 1:250 |
| | added to pellet | | | |
| Fungi | 5 ml TE buffer | 5 μl | 245μ | 1:250 |
| | sdded to pellet | | | |
| Viruses | 2 ml culture | 2 μl | 198 μl | 1:100 |
| | from ATCC | | | |

The Applicants' studies suggest that spiking an ATCC culture (pellet suspended in 5 ml of medium or buffer) diluted at 1:50 simulates the concentration of bacterial and fungal pathogens (i.e., 250-fold dilution of ATCC culture) and 1:100 dilution simulates the viral pathogen (100-fold dilution of ATCC culture) in the clinical sample.

Studying the Stability of the Pathogen

For validation studies, Copan UTM-RT transport medium (Lot #A 303CS02) as provided by the manufacturer was pooled in a sterile bottle. Based upon the simulated dilutions described above for each pathogen, the following cocktails were prepared:

TABLE 4

Per vial (A, B, & C)

| | | | μl Copan |
|---|---|---|---|
| Cocktail | Pathogens | μl Pathogen* | UTM-RT |
| 1 | Candida albicans | 80 μl | 3840 μl |
| | Neisseria | 80 μl | |
| | gonorrhoeae | | |
| 2 | Candida | 80 μl | 3800 μl |
| | parapsilosis | | |
| | Chlamydia | 80 μl | |
| | trachomatis | | |
| | Herpes Simplex | 40 μl | |
| | Virus-1 | | |
| 3 | Candida glabrata | 80 μl | 3800 μl |
| | Herpes Simplex | 40 μl | |
| | Virus-2 | | |
| | Trichomonas | 80 μl | |
| | vaginalis | | |
| 4 | Candida | 80 μl | 3760 μl |
| | tropicalis | | |
| | Mobiluncus | 80 μl | |
| | mulieris | | |
| | Ureaplasma | 80 μl | |
| | urealyticum | | |
| 5 | Bacteriodes | 80 μl | 3840 μl |
| | fragilis | | |
| | Mycoplasma | 80 μl | |
| | hominis | | |
| 6 | Gardnerella | 80 μl | 3840 μl |
| | vaginalis | | |
| | Haemophilis | 80 μl | |
| | ducreyi | | |

*Dilution prepared for each pathogen as detailed in Table 4.

Each cocktail was prepared in triplicate (15 ml tubes) and designated A, B, or C. Pathogen culture solution was added to obtain desired concentration which mimics the pathogen load in a positive clinical sample (1:250-fold dilution for ATCC bacterial and fungal culture and 1:100-fold dilution for ATCC virus culture). 500 μl of the above mix was transferred to three separate microcentrifuge tubes labeled Day 0 to 5.

Inoculated media vials of each cocktail were incubated at room temperature. At 24 hour intervals starting with Day 0 through Day 5, three microcentrifuge tubes were transferred to −20° C. storage. Aliquots from each vial were extracted for DNA by standard laboratory procedures after Day 5. Conventional and real-time PCR reactions for each pathogen on the appropriate cocktail followed. The summary of results is as follows:

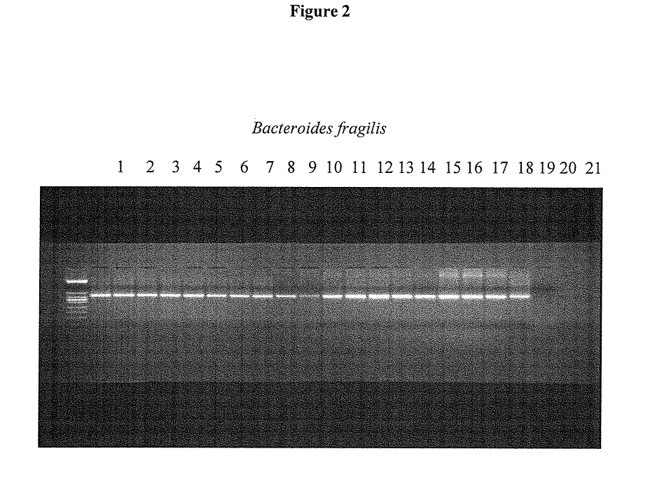

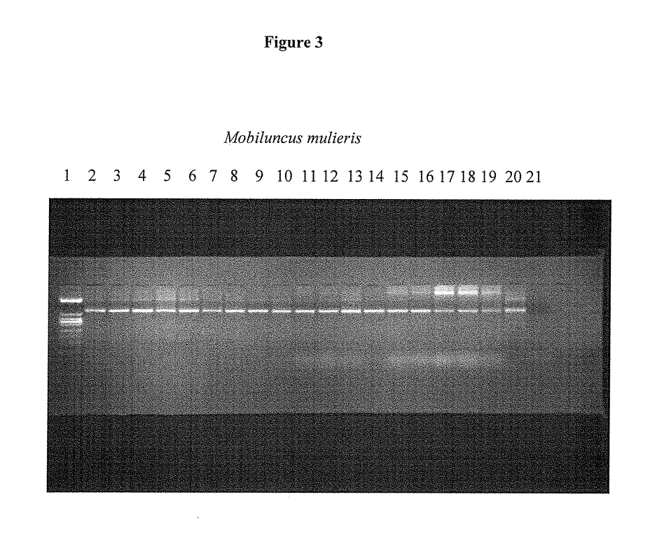

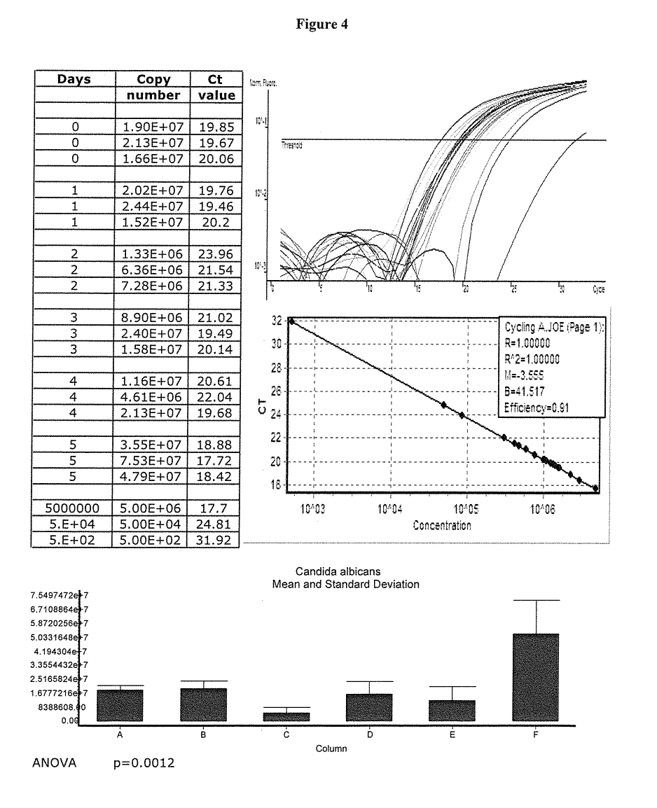

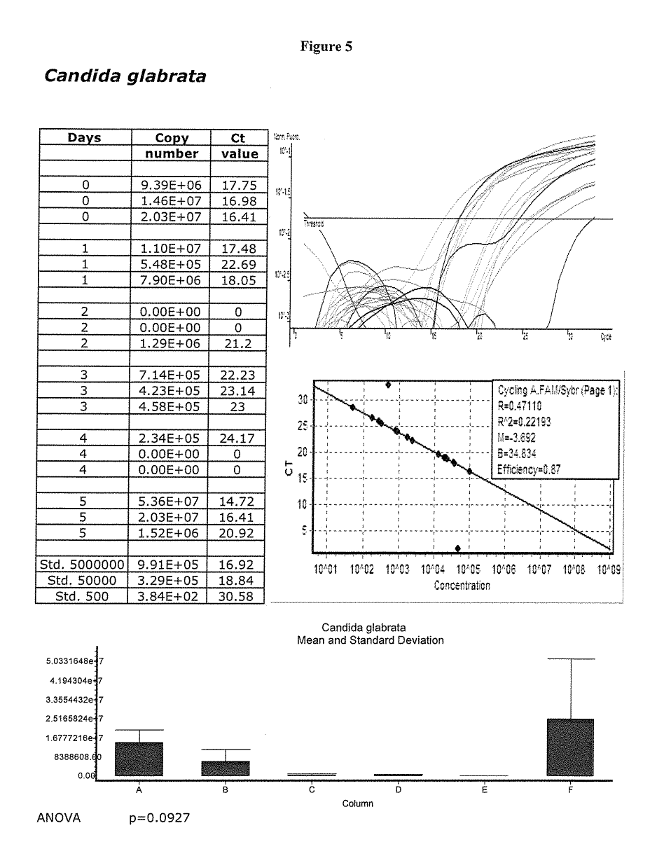

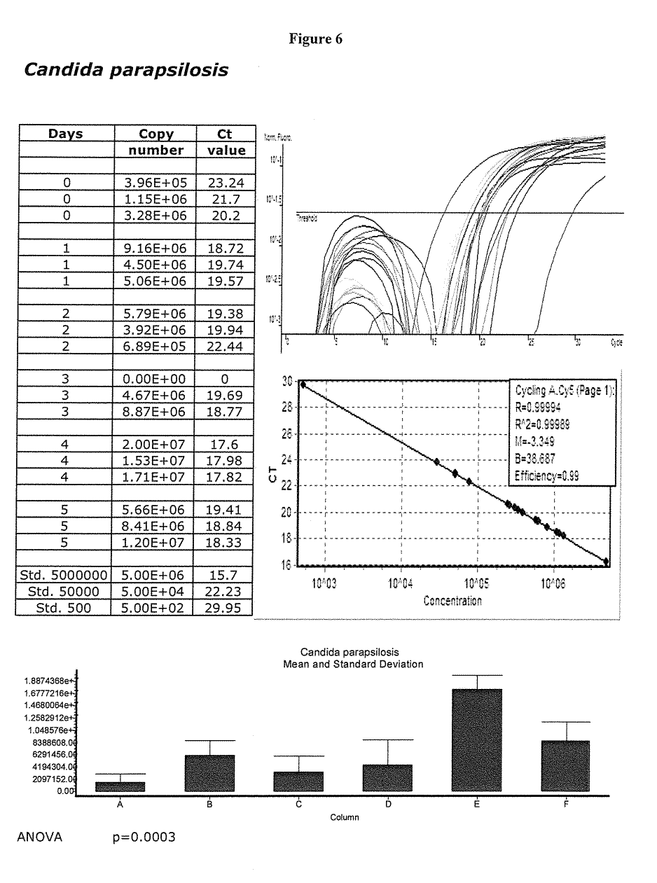

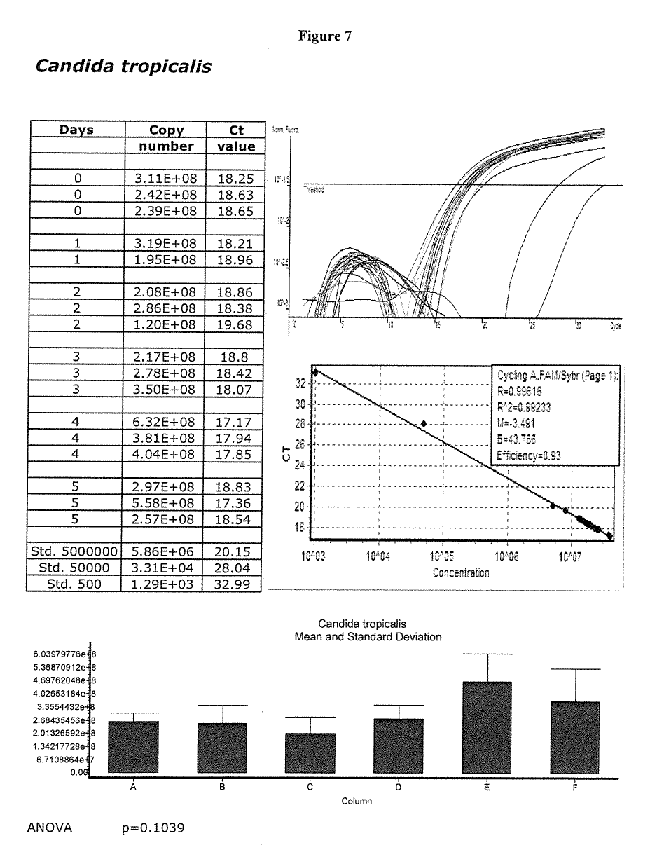

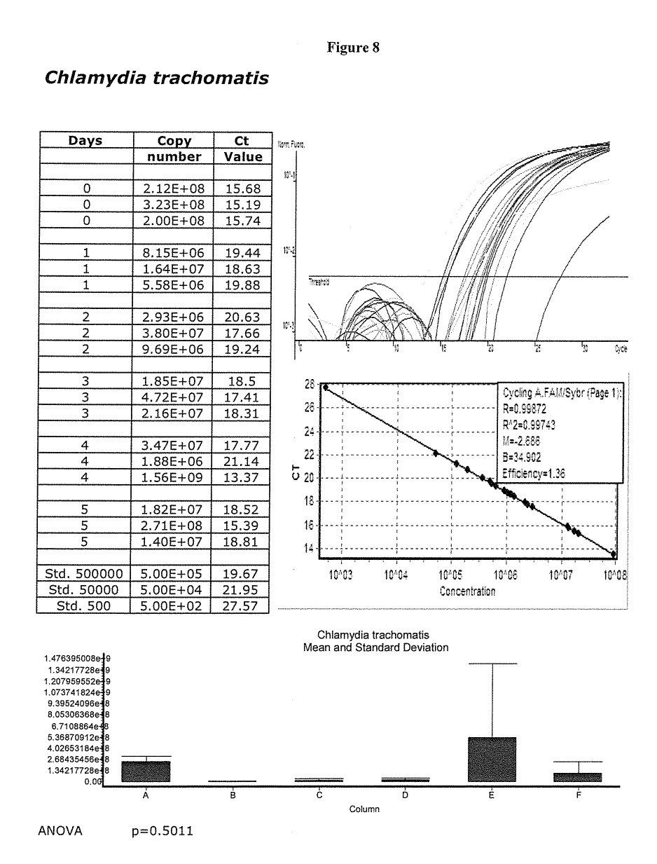

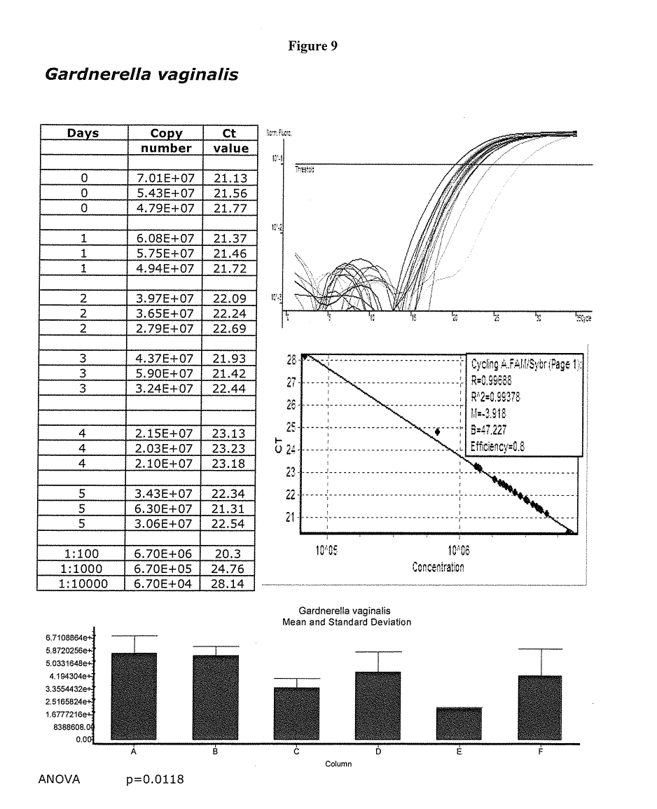

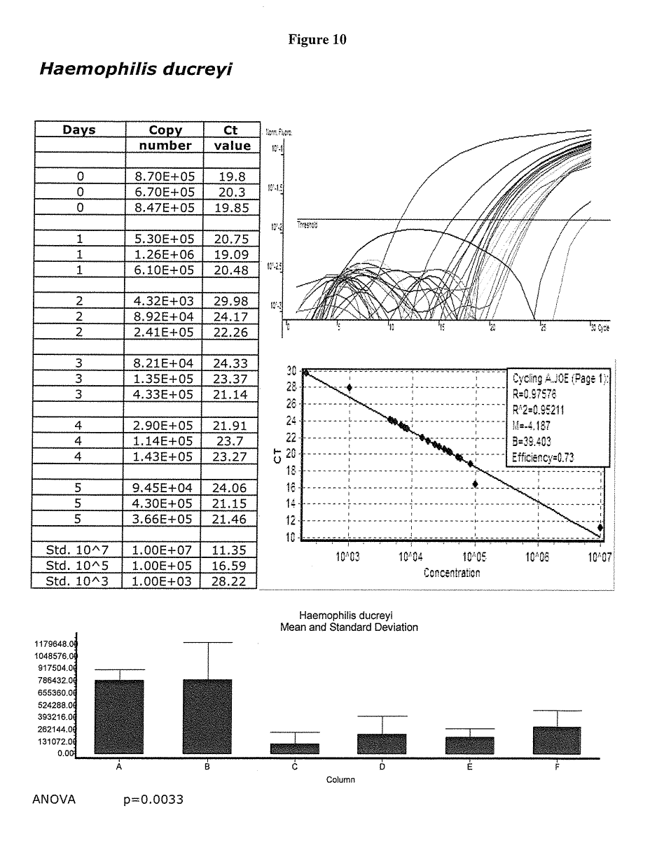

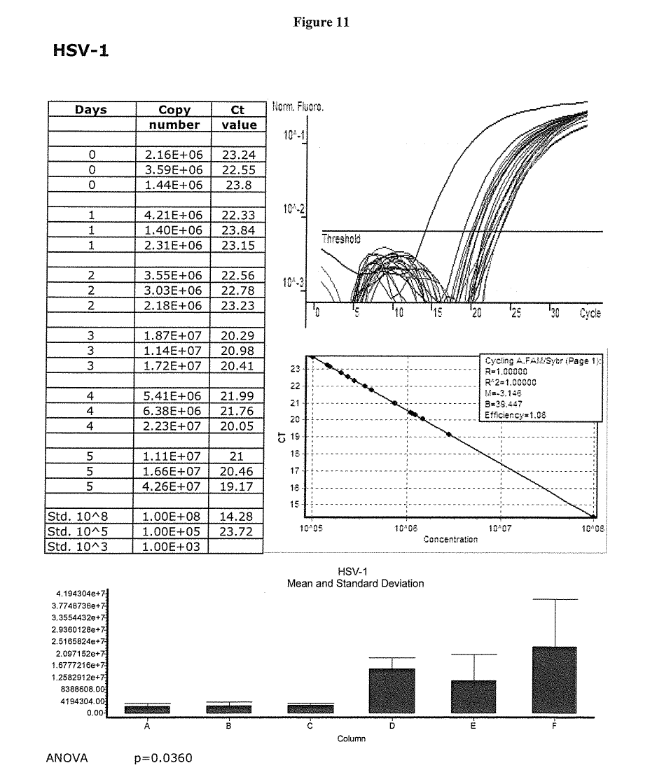

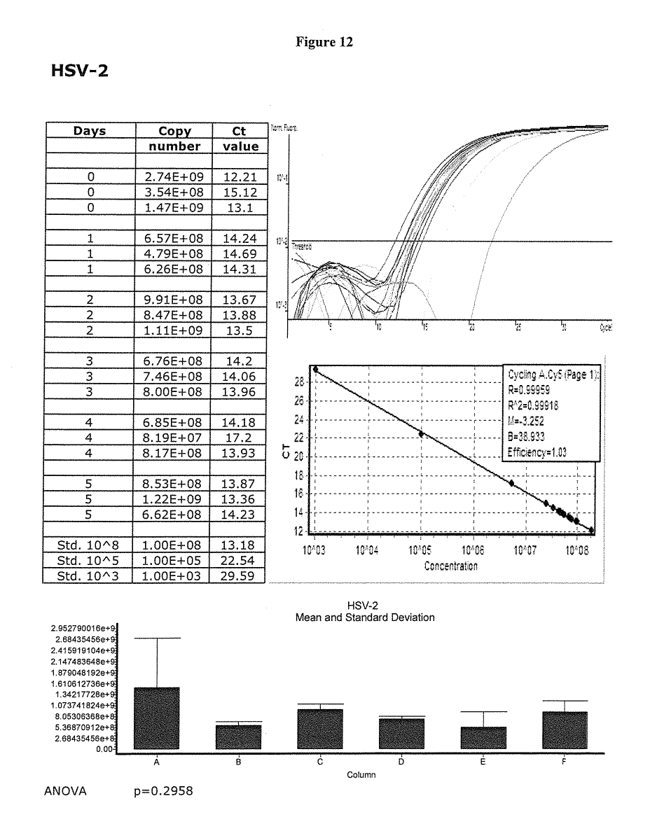

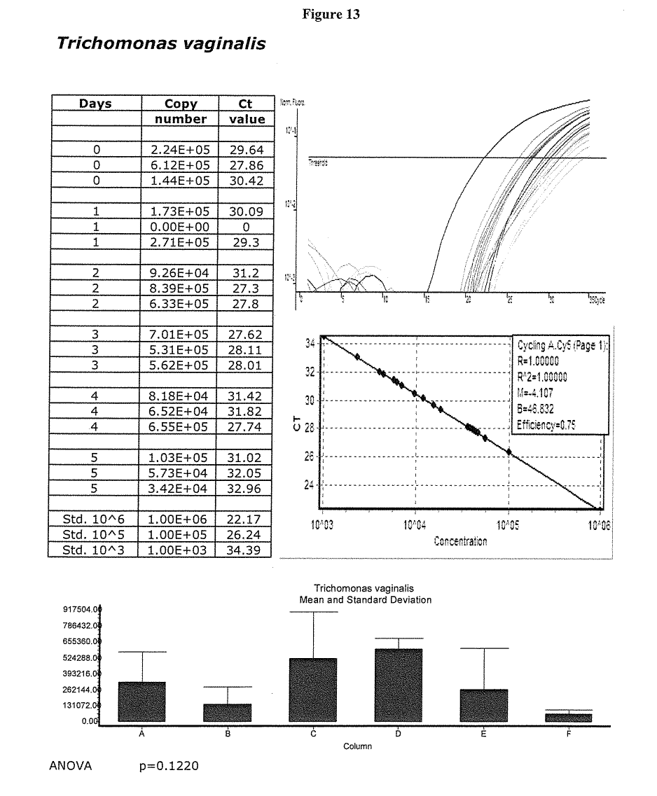

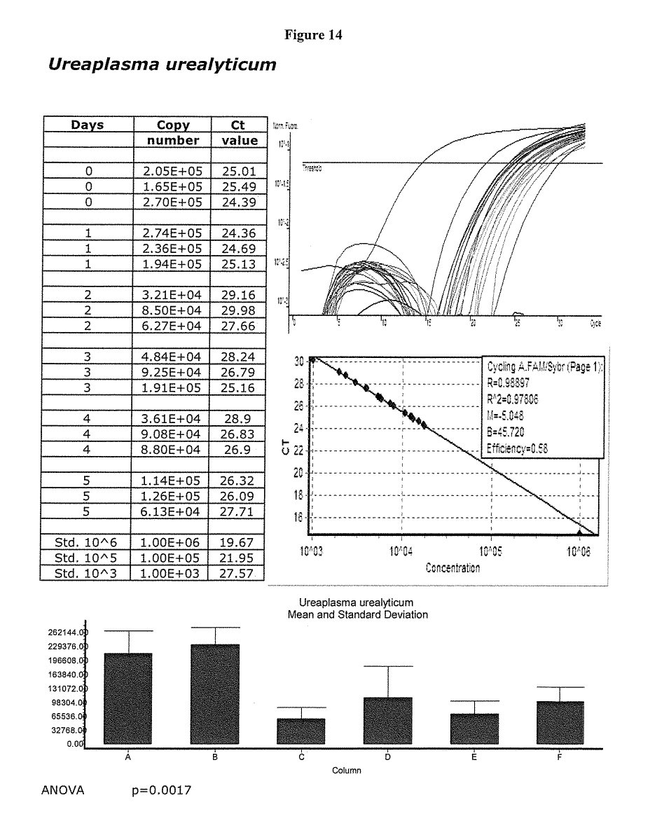

TABLE 5

| | | | # Positive Time |
|---|---|---|---|
| | | | Pts./ |
| | | | # Specimens |
| | Pathogen | Type of PCR | Tested |
| 1 | Bacteriodes | Conventional | 18/18 (see |
| | fragilis | PCR | FIG. 2) |
| 2 | Candida | Real-time | 18/18 (see |
| | albicans | PCR | FIG. 4) |
| 3 | Candida | Real-time | 18/18 (see |
| | glabrata | PCR | FIG. 5) |
| 4 | Candida | Real-time | 18/18 (see |
| | parapsilosis | PCR | FIG. 6) |
| 5 | Candida | Real-time | 18/18 (see |
| | tropicalis | PCR | FIG. 7) |
| 6 | Chlamydia | Real-time | 18/18 (see |
| | trachomatis | PCR | FIG. 8) |
| 7 | Gardnerella | Real-time | 18/18 (see |
| | vaginalis | PCR | FIG. 9) |
| 8 | Haemophilia | Real-time | 18/18 (see |
| | ducreyi | PCR | FIG. 10) |
| 9 | Herpes Simples | Real-time | 18/18 (see |
| | Virus-1 | PCR | FIG. 11) |
| 10 | Herpes Simples | Real-time | 18/18 (see |
| | Virus-2 | PCR | FIG. 12) |
| 11 | Mobiluncus | Conventional | 18/18 (see |
| | mulieris | PCR | FIG. 3) |
| 12 | Mycoplasma | Conventional | 6/6 |
| | hominis | PCR | |
| 13 | Neisseria | Real-time | 18/18 |
| | gonorrhoeae | PCR | |
| 14 | Trichomonas | Real-time | 18/18 (see |
| | vaginalis | PCR | FIG. 13) |
| 15 | Ureaplasma | Real-time | 18/18 (see |
| | urealyticum | PCR | FIG. 14) |

### Example II

### DNA Extraction from Transport Media

For DNA extraction, see, e.g., Goessens et al., 1995, Influence of volume of sample processed on detection of Chlamydia trachomatis in urogenital samples by PCR. Journal of Clinical Microbiology 33:251-253.

The following steps outline the procedure to isolate and purify DNA from transport media. The specimen is submitted as a self-contained unit with transport media.

Proteinase K: 100 μl Tris (pH 7.5), 4.9 ml ddH₂O, 5 ml glycerol. Dissolve well and store at −20° C. as 500 μl aliquots. ps 10% SDS: 10 g SDS in 100 ml of ddH₂O.

### Equipment

Disposable pipette tips

Disposable transfer pipette

Laboratory timer

1.5 ml microcentrifuge tube

1.6 55° C. water bath

Pipettes to deliver a range of 1-1000 μl

### Procedure

1. Mix the swab thoroughly in the transport media.
2. Pipette 470 μl of transport media into a labeled microcentrifuge tube.
3. Add 25 μl of 10% SDS and 12 μl of Proteinase K. Mix well.
4. Incubate for 2 hours in 55° C. water bath.
5. After 2 hours, place 200 μl of Tris saturated phenol and 200 μl of chloroform:isoamyl alcohol (24:1) in the tube. Shake the tube to mix the layers.
6. Centrifuge at 14,000 rpm for 5 minutes at room temperature. This will separate the layers.
7. Remove the top chloroform layer (containing the DNA) being careful not to pipette any of the bottom or middle layers. Place this into another labeled microcentrifuge tube. The first tube containing the remaining layers may be discarded.
8. To this new tube add 0.1×volume of 3 M sodium acetate. Also add 2×volumes of cold 100% ethanol. Vortex and place in −20° C. overnight.
9. Centrifuge the tube at 14,000 rpm at 4° C. for 10 minutes. This will pellet the precipitated DNA.
10. Remove and discard the supernatant. Add 1000 μl of 70% ethanol to wash the pellet. Slightly resuspend the pellet.
11. Centrifuge the tube again at 14,000 rpm at 4° C. for 5 minutes to form a pellet.
12. Place the tube with the top open into the CentriVap (Labconco, Kansas City, Mo.). Spin at 35° C. for approximately 15 minutes. Spin until the pellet is dry, being very careful not to overdry.
13. Resuspend the pellet in 20 μl of ddH2O.
14. Quantitate the DNA using a spectrophotometer.

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following

What is claimed is:

1. A method of handling a plurality of clinical samples and managing information associated therewith by a clinical laboratory for reporting a sum of diagnostic results for each sample comprising:
(a) receiving a plurality of primary gynecological swab samples, each primary gynecological swab sample in said plurality of gynecological swab samples having identity and test requisition information associated therewith wherein the test requisition information indicates a test for at least one microbiological causative agent from a plurality of microbiological causative agents listed in said test requisition, wherein said plurality of causative agents listed in said test requisition comprise microbiological species associated with pathological gynecological conditions;
(b) entering said requisition information into a computer system to create a requisition file for each said primary gynecological swab sample;
(c) processing said requisition information in said computer system to create a list of tests for each said microbiological causative agent;
(d) dispensing an aliquot corresponding to each said primary gynecological swab sample into one or more individual vessels, to create one or more secondary samples, each secondary sample corresponding to each said indicated test;
(e) assembling a general supply of master reagent mix for each said indicated test;
(f) combining an aliquot of a master reagent mix for each said indicated test with each corresponding secondary sample to produce a diagnostic test reaction for each said secondary sample;
(g) incubating each said diagnostic test reaction;
(h) performing a polymerase chain reaction (PCR) to determine the presence or absence of a certain product of each said diagnostic test reaction to produce a result;
(i) recording said result of each said PCR by means of said computer system;
(j) entering said result of each said PCR reaction derived from each said primary gynecological swab sample into said requisition file for each said sample on said computer system, thereby producing a sum of results of all tests for each said primary gynecological swab sample; and
(k) reporting said results to a physician within 24-48 hours of receiving said primary gynecological swab sample.

2. The method according to claim 1 wherein said microbiological species comprise Candida dubliniensis, Candida lusitaneae, or both.

3. The method according to claim 1 wherein said microbiological species comprise at least one of the following: Candida dubliniensis, Candida lusitaneae, Atopobium vaginae Lymphogranuloma venereum, erythromycin-resistant Streptococcus agalactiae or clindamycin-resistant Streptococcus agalactiae.

4. The method according to claim 1 wherein said microbiological species comprise at least one of the following: Candida dubliniensis, Candida lusitaneae, Atopobium vaginae Lymphogranuloma venereum, erythromycin-resistant Streptococcus agalactiae clindamycin-resistant Streptococcus.agalactiae, Bacteroides fragilis, Candida albicans, Candida glabrata, Candida parapsilosis, Candida tropicalis, Chlamydia trachomatis, Gardnerella vaginalis, Haemophilus ducreyi, Herpes simplex virus subtype 1, Herpes simplex virus subtype 2, Human papillomavirus (HPV), Mobiluncus mulieris, Mobiluncus curtisii, Molluscum contagiosum Virus, Mycoplasma genitalium, Mycoplasma hominis, Neisseria gonorrhoeae, Treponema pallidum, Trichomonas vaginalis, Ureaplasma urealyticum, Streptococcus agalactiae, Candida krusei, HPV-16, HPV-18, HPV-31, HPV-33, HPV-35, HPV-39, HPV-45, HPV-51, HPV-52, HPV-56, HPV-58, HPV-59, HPV-66, HPV-68, HPV-30 6/11, HPV-42, HPV-43, or HPV-44.

5. The method according to claim 1 wherein said microbiological species comprise at least two of the following: Candida dubliniensis, Candida lusitaneae, Atopobium vaginae Lymphogranuloma venereum, erythromycin-resistant Streptococcus agalactiae clindamycin-resistant Streptococcus agalactiae., Bacteroides fragilis, Candida albicans, Candida glabrata, Candida parapsilosis, Candida tropicalis, Chlamydia trachomatis, Gardnerella vaginalis, Haemophilus ducreyi, Herpes simplex virus subtype 1, Herpes simplex virus subtype 2, Human papillomavirus (HPV), Mobiluncus mulieris, Mobiluncus curtisii, Molluscum contagiosum Virus, Mycoplasma genitalium, Mycoplasma hominis, Neisseria gonorrhoeae, Treponema pallidum, Trichomonas vaginalis, Ureaplasma urealyticum, Streptococcus agalactiae, Candida krusei, HPV-16, HPV-18, HPV-31, HPV-33, HPV-35, HPV-39, HPV-45, HPV-51, HPV-52, HPV-56, HPV-58, HPV-59, HPV-66, HPV-68, HPV-30 6/11, HPV-42, HPV-43, or HPV-44.

6. The method according to claim 1 wherein said microbiological species comprise at least five of the following: Candida dubliniensis, Candida lusitaneae, Atopobium vaginae Lymphogranuloma venereum, erythromycin-resistant Streptococcus agalactiae clindamycin-resistant Streptococcus agalactiae, Bacteroides fragilis, Candida albicans, Candida glabrata, Candida parapsilosis, Candida tropicalis, Chlamydia trachomatis, Gardnerella vaginalis, Haemophilus ducreyi, Herpes simplex virus subtype 1, Herpes simplex virus subtype 2, Human papillomavirus (HPV), Mobiluncus mulieris, Mobiluncus curtisii, Molluscum contagiosum Virus, Mycoplasma genitalium, Mycoplasma hominis, Neisseria gonorrhoeae, Treponema pallidum, Trichomonas vaginalis, Ureaplasma urealyticum, Streptococcus agalactiae, Candida krusei, HPV-16, HPV-18, HPV-31, HPV-33, HPV-35, HPV-39, HPV-45, HPV-51, HPV-52, HPV-56, HPV-58, HPV-59, HPV-66, HPV-68, HPV-30 6/11, HPV-42, HPV-43, or HPV-44.

7. The method according to claim 1 wherein said microbiological species are at least five species selected from the group consisting of Candida dubliniensis, Candida lusitaneae, Atopobium vaginae Lymphogranuloma venereum, erythromycin-resistant Streptococcus agalactiae clindamycin-resistant Streptococcus.agalactiae, Bacteroides fragilis, Candida albicans, Candida glabrata, Candida parapsilosis, Candida tropicalis, Chlamydia trachomatis, Gardnerella vaginalis, Haemophilus ducreyi, Herpes simplex virus subtype 1, Herpes simplex virus subtype 2, Human papillomavirus (HPV), Mobiluncus mulieris, Mobiluncus curtisii, Molluscum contagiosum Virus, Mycoplasma genitalium, Mycoplasma hominis, Neisseria gonorrhoeae, Treponema pallidum, Trichomonas vaginalis, Ureaplasma urealyticum, Streptococcus agalactiae, Candida krusei, HPV-16, HPV-18, HPV-31, HPV-33, HPV-35, HPV-39, HPV-45, HPV-51, HPV-52, HPV-56, HPV-58, HPV-59, HPV-66, HPV-68, HPV-30 6/11, HPV-42, HPV-43, or HPV-44.

8. The method according to claim 1 wherein the aliquot corresponding to each said primary gynecological swab sample in step (d) contains nucleic acid that has been extracted and quantitated from each primary gynecological swab sample.

9. The method according to claim 1 wherein the presence or absence of a product of at least one reaction is optically monitored and electronically recorded by means of the computer system.

10. The method according to claim 1 for identifying at least two different microbiological causative agents in at least one primary gynecological swab sample.

11. The method according to claim 1 for identifying at least three different microbiological causative agents in at least one primary gynecological swab sample.

12. The method according to claim 1 wherein at least one diagnostic test reaction comprises a real-time Polymerase Chain Reaction (PCR).

13. The method according to claim 1 wherein the results are reported within about 24-30 hours of receiving the sample.

14. A method of handling a plurality of clinical samples and managing information associated therewith by a clinical laboratory for reporting a sum of diagnostic results for each sample comprising:
(a) receiving a plurality of primary gynecological swab samples, each primary gynecological swab sample in said plurality of gynecological swab samples having identity and test requisition information associated therewith wherein the test requisition information indicates a test for at least one microbiological causative agent from a plurality of microbiological causative agents listed in said test requisition, wherein said plurality of causative agents listed in said test requisition comprises Ureaplasma parvum,
(b) entering said requisition information into a computer system to create a requisition file for each said primary gynecological swab sample;
(c) processing said requisition information in said computer system to create a list of tests for each said microbiological causative agent;
(d) dispensing an aliquot corresponding to each said primary gynecological swab sample into one or more individual vessels, to create one or more secondary samples, each secondary sample corresponding to each said indicated test;
(e) assembling a general supply of master reagent mix for each said indicated test;
(f) combining an aliquot of a master reagent mix for each said indicated test with each corresponding secondary sample to produce a diagnostic test reaction for each said secondary sample;
(g) incubating each said diagnostic test reaction;
(h) performing a polymerase chain reaction (PCR) to determine the presence or absence of a certain product of each said diagnostic test reaction to produce a result;
(i) recording said result of each said PCR by means of said computer system;
(j) entering said result of each said PCR reaction derived from each said primary gynecological swab sample into said requisition file for each said sample on said computer system, thereby producing a sum of results of all tests for each said primary gynecological swab sample; and
(k) reporting said results to a physician within 24-48 hours of receiving said primary gynecological swab sample.
